(19) United States
(12) Patent Application Publication
(10) Pub. No.: US 2005/0038481 A1
(43) Pub. Date: Feb. 17, 2005

(54) Evaluating ventricular synchrony based on phase angle between sensor signals

(76) Inventors: Edward Chinchoy, Minneapolis, MN (US); D. Curtis Deno, Andover, MN (US); Yong K. Cho, Maple Grove, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/638,883

(22) Filed: Aug. 11, 2003

(51) Int. Cl. A61N 1/365

(52) U.S. Cl. 607/17

(57) ABSTRACT

An implantable medical device evaluates ventricular synchrony by determining a phase angle between at least two sensor signals that reflect mechanical contraction of the ventricles. In exemplary embodiments, two intracardiac impedance signals associated with the right and left ventricles, respectively, with two points within either of the left and right ventricles, or with both the left and right ventricles relative to a reference point, are processed. In such embodiments, fundamental frequency phases of each of the impedance signals may be compared to determine the phase angle between the signals. In some embodiments, the signals are used to dynamically adjust one or more timing intervals, such as a V-V timing interval, for delivery of cardiac resynchronization therapy (CRT) pacing. In such embodiments, the one or more timing intervals are periodically adjusted to reduce or possibly eliminate ventricular dysynchrony as indicated by the phase angle between the sensor signals.

### TECHNICAL FIELD

The invention relates to implantable medical devices, and more particularly, to implantable medical devices that provide cardiac pacing.

### BACKGROUND OF THE INVENTION

Ventricular dysynchrony is characterized by non-simultaneous ventricular contractions. In particular, movement of an apical region of the heart is out of synch with movement of the left-ventricular free wall of the heart. Ventricular dysynchrony is caused by conduction delays within the ventricles. The conduction delays are caused by, for example, bundle branch blocks or infracted tissue within the myocardium. Typically, ventricular dysynchrony is assessed in a clinical setting via echocardiography.

As a result of ventricular dysynchrony, the heart pumps blood less efficiently resulting in symptoms of heart failure. Consequently, some patients diagnosed with ventricular dysynchrony receive cardiac resynchronization therapy (CRT) pacing. CRT pacing therapies typically involve delivery of pacing pulses to both ventricles with an interventricular, i.e., V-V, interval therebetween in an attempt to “resynchronize” the ventricular contractions resulting from delivery of the pacing pulses. V-V intervals often vary depending on the underlying etiology of the disease including location and severities of electrical conduction disorders and infarcts.

Over time, the condition or disease of a patient can change, rendering a V-V interval selected by a physician on the basis of an acute echocardiograph inappropriate. Existing techniques for dynamically adjusting the V-V interval to meet changing patient conditions have involved adjusting the V-V interval to maximize cardiac performance as indicated by a physiological parameter, such as a cardiac pressure. These techniques, however, are only able to indirectly evaluate ventricular synchrony. Further, the physiological parameters monitored can vary over time due to factors other than changes in the dysynchrony of the ventricles, potentially leading to selection of an inappropriate V-V interval.

### SUMMARY OF THE INVENTION

In general, the invention is directed to techniques for evaluating ventricular synchrony based on the phase angle between sensor signals. In exemplary embodiments, a medical device according to the invention, such as an implantable medical device (IMD), delivers cardiac resynchronization therapy (CRT) pacing, and adjusts one or more timing intervals for the CRT pacing, such as a V-V interval, based on the phase angle. Such a medical device can dynamically adjust the timing interval(s) to reduce, or possibly eliminate, ventricular dysynchrony.

The medical device includes pacing electrodes to deliver pacing pulses to one or more sites for each of the left and right ventricles, or to one or more sites for either of the left and right ventricles. In some embodiments, the sensor signals are impedance signals detected by left and/or right ventricular electrodes, atrial electrodes, housing electrodes of an IMD, or any combination thereof. In other embodiments, the sensor signals are ventricular acceleration, intracardiac pressure, or oximetery signals that reflect contraction of the ventricles. In exemplary embodiments, two signals reflecting right and left ventricular contractions, respectively, are compared. The phase angle between the signals can be determined using a variety of techniques, including comparison of fiducial points within the signals and comparison of the fundamental frequency phase of the signals.

In one embodiment, the invention is directed to a method in which signals that reflect contractions of at least one ventricle of a heart are received from respective sensors implanted within a patient. The signals are processed to determine a phase angle between the signals, and at least one interval for ventricular pacing is adjusted based on the phase angle.

In another embodiment, the invention is directed to a medical device that includes sensors implanted within a patient to generate respective signals that reflect contractions of at least one ventricle of a heart of the patient, and a processor to determine a phase angle between the signals. The processor adjusts at least one interval for ventricular pacing based on the phase angle.

In another embodiment, the invention is directed to a computer-readable medium containing instructions. The instructions cause a programmable processor to determine a phase angle between signals that reflect contractions of at least one ventricle of a heart of a patient, and adjust at least one interval for ventricular pacing based on the phase angle. Each of the signals is generated by a respective sensor implanted within the patient.

In another embodiment, the invention is directed to a medical device comprising means for generating signals that reflect contractions of at least one ventricle of a heart, and means for determining a phase angle between the signals and adjusting at least one interval for ventricular pacing based on the phase angle. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an exemplary implantable medical device that evaluates ventricular synchrony based on a phase angle between signals according to the invention.

FIG. 2 is a functional block diagram of the implantable medical device of FIG. 1.

FIG. 3 is a timing diagram illustrating exemplary signals that reflect contractions of a right and left ventricles of a heart, respectively.

FIG. 4 is a flow chart illustrating an example operation of the IMD of FIG. 1 to adjust delivery of cardiac resynchronization therapy based on a phase angle between signals.

### DETAILED DESCRIPTION OF THE EMBODIMENTS

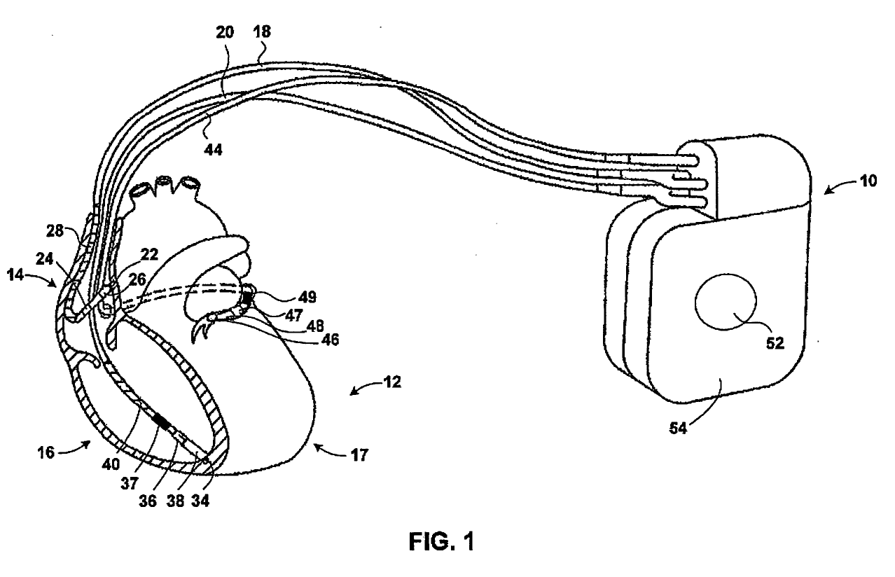

FIG. 1 depicts an exemplary implantable medical device (IMD) 10 that evaluates ventricular synchrony according to the invention. In the illustrated example, IMD 10 takes the form of a multi-chamber implantable cardioverter-defibrillator (ICD). IMD 10 is implanted within a patient (not shown), and includes leads 18, 20 and 24 that extend into a heart 12 of the patient.

Right trial (RA) lead 18 extends into the right atrium 14 of heart 12. Right ventricular (RV) lead 20 extends through the right atrium 14, and into the right ventricle 16 of heart 12. Left ventricular (LV) coronary sinus lead 44 extends through the right atrium 14, and into the coronary sinus of heart 12 to a point adjacent to the free wall of left ventricle 17 of heart 12.

Leads 18, 20 and 44 include electrodes for sensing electrical activity attendant to the depolarization and repolarization of heart 12, and for delivering pacing pulses to heart 12. In the illustrated embodiment, bipolar electrode pairs 22 and 24, 34 and 36, and 46 and 47 are located adjacent distal end of leads 18, 20 and 44, respectively. In exemplary embodiments, electrodes 22, 34 and 46 take the form of ring electrodes, and electrodes 24, 36 and 47 take the form of extendable helix tip electrodes mounted retractably within insulative electrode heads 26, 38 and 48, respectively.

In the illustrated embodiment, IMD 10 also includes an indifferent housing electrode 52, formed integrally with an outer surface of the hermetically sealed housing 54 of IMD 10. In such embodiments, IMD 10 is capable of using any of electrodes 22, 24, 34, 36, 46 and 47 for unipolar sensing or pacing in combination with housing electrode 48. In some embodiments, IMD 10 delivers cardioversion and/or defibrillation shocks to heart 12 via coil electrodes 28 and 40 of leads 18 and 20, respectively. Lead 44 may also include one or more coil electrodes (not shown).

As will be described in greater detail below, IMD 10 processes sensor signals to evaluate ventricular synchrony. The signals reflect mechanical contractions of ventricles 16 and 17. In exemplary embodiments, IMD 10 processes two signals, each of the signals reflecting a mechanical contraction of one of the right and left ventricles. In other embodiments, IMD 10 processes two or more signals reflecting contractions of one of ventricles 16 and 17. In still other embodiments, IMD 10 processes additional signals that are associated with reference points.

IMD 10 determines a phase angle between the signals and compares the phase angle to a predetermined target value. If the phase angle does not equal the target value, this indicates ventricular dysynchrony. However, if the phase angle is equal to the target value, this indicates ventricular synchrony. In some embodiments, the predetermined target value is zero. In embodiments where IMD 10 processes signals associated with a reference point, determination of phase angles between signals associated with ventricles 16 and 17 and the reference point rather than between two ventricular signals can reduce the impact of noise on the phase angle determination. Techniques that may be used by IMD 10 to determine the phase angle between the signals will be described in greater detail below.

In some embodiments, the sensor signals comprise intracardiac impedance signals received from combinations of electrodes 22, 24, 34, 36, 46 and 47. IMD 10 outputs current between two or more of the electrodes, and measures the voltage between two or more of the electrodes to determine an intracardiac impedance. In exemplary embodiments, the current is output in the form of biphasic current pulses.

To generate a right ventricular impedance signal, IMD 10 can, for example, apply current and detect voltage across electrode 34 and either of electrodes 24 and 52. To generate a three-terminal right ventricular impedance signal, IMD 10 can, for example, apply current across electrode 36 and either of electrodes 34 and 24, and detect voltage across electrode 36 and the other of electrodes 34 and 24.

To generate a left ventricular impedance signal, IMD 10 can, for example, apply current and detect voltage across electrode 46 and either of electrodes 24 and 52. To generate a three-terminal left-ventricular signal, IMD 10 can, for example, apply current across electrode 47 and either of electrodes 46 or 24, and detect voltage across electrode 47 and the other of electrodes 46 or 24.

To reduce the presence of noise in impedance signals detected as discussed above, a reference point common to two or more signals, such as electrode 24 in right atrium 14, can be selected. In exemplary embodiments, IMD 10 detects the voltage associated with the right ventricular impedance signal across electrode 34 and electrode 24. Since electrode 24 is a reference point common to both signals, IMD 10 further detects the voltage associated with the left ventricular impedance signal across electrode 46 and electrode 24. Using this method, noise on both signals is reduced and can possibly be eliminated. In other embodiments, electrode 54 of the housing of IMD 10 serves as the reference point when detecting left and right impedance signals.

Another exemplary technique for generating right and left ventricular impedance signals is to apply current from electrode 34 to electrode 46. The right ventricular impedance is measured by measuring the voltage between electrodes 34 and 36. The left ventricular impedance is measured by measuring the voltage across electrode 46 and either of electrodes 47, 52, 36 or 24.

In other embodiments, IMD 10, as shown in FIG. 1, includes sensors 37 and 49 that generate signals that reflect contractions of ventricles 16 and 17. In the illustrated embodiment, sensors 37 and 49 are located distally on leads 20 and 44, and generate signals that reflect contractions of the right and left ventricles, respectively. In various embodiments, sensors 37 and 49 comprise one of accelerometers, pressure sensors, and oximeters. In some embodiments, sensors 37 and 49 are capacitive absolute pressure sensors, as described in U.S. Pat. No. 5,564,434 to Halperin, et al., hereby incorporated by reference herein in its entirety, piezoelectric crystals, or piezoresistive pressure transducers.

IMD 10 provides cardiac resynchronization therapy (CRT) pacing to the patient. In exemplary embodiments, IMD 10 delivers pacing pulses to both of ventricles 16 and 17, and the deliveries of pacing pulses to left and right ventricles 16 and 17 are separated by an interventricular delay, i.e. a V-V delay. Either the right or left ventricular pacing pulse may be delivered first, or, in some embodiments, the V-V timing interval is zero and the pulses are delivered simultaneously. In some embodiments, the V-V timing interval defines an interval between an intrinsic depolarization of one ventricle and a delivery of a pacing pulse to another ventricle. Further, in some embodiments, the V-V timing interval is defined as the difference between first and second intervals that are between an atrial depolarization and delivery of pacing pulses to each of ventricles 16 and 17.

IMD 10 provides CRT pacing to synchronize contraction of the ventricles. Because a phase angle between the sensor signals that is not equal to the predetermined target value, such as zero, indicates non-synchronous ventricular contraction, IMD 10 adjusts one or more timing intervals, such as the V-V timing interval described above, based on a determination that the phase angle is not equal to the target value. In this manner, exemplary embodiments of IMD 10 provide one or more dynamic timing intervals that are more effective in maintaining proper ventricular synchrony.

The configuration of IMD 10 and leads 18, 20 and 44 and location of electrodes 22, 24, 24, 26, 46 and 47 illustrated in FIG. 1 is merely exemplary. In various embodiments, IMD 10 is coupled to any number of leads that extend to a variety of positions within or outside of heart 12. For example, in some embodiments, IMD 10 is coupled to a lead that extends to the left atrium of heart 12, or epicardial leads that extend to any position on an exterior surface of heart 12. In some embodiments, multiple leads extend to one or both of the ventricles 16 and 17.

Further, medical devices evaluate ventricular synchrony according to some embodiments of the invention are not implanted in the patient, but instead are coupled to subcutaneous leads that extend through the skin of the patient to a variety of positions within or outside of heart 12. In some embodiments, a clinician can use a medical device according to the invention acutely to evaluate ventricular synchrony. In some embodiments, the clinician adjusts one or more timing intervals, such as the V-V timing interval, based on a determined phase angle.

As discussed above, in some embodiments, IMD 10 does not include sensors 37 and 49, but instead determines the phase angle between cardiac impedance signals received from combinations of electrodes 22, 24, 34, 36, 46 and 47. In other embodiments, IMD 10 uses both impedance signals and signals generated by sensors 37 and 49. IMD 10 can include any number of sensors and can evaluate any number of sensor signals.

Sensors 37 and 49 need not be carried on leads 20 and 44. In some embodiments, sensors 37 and 49 are carried on leads that are implanted in addition to leads 20 and 44. Sensors 37 and 49 can be located anywhere within or outside the heart suitable for generation of a signal that reflects mechanical contraction of ventricles 16 and 17. For example, the lead to which sensor 47 is carried on can be inserted into the left ventricle by puncturing through either the outer left ventricular wall, the apical region, or through the septum from right ventricle 16.

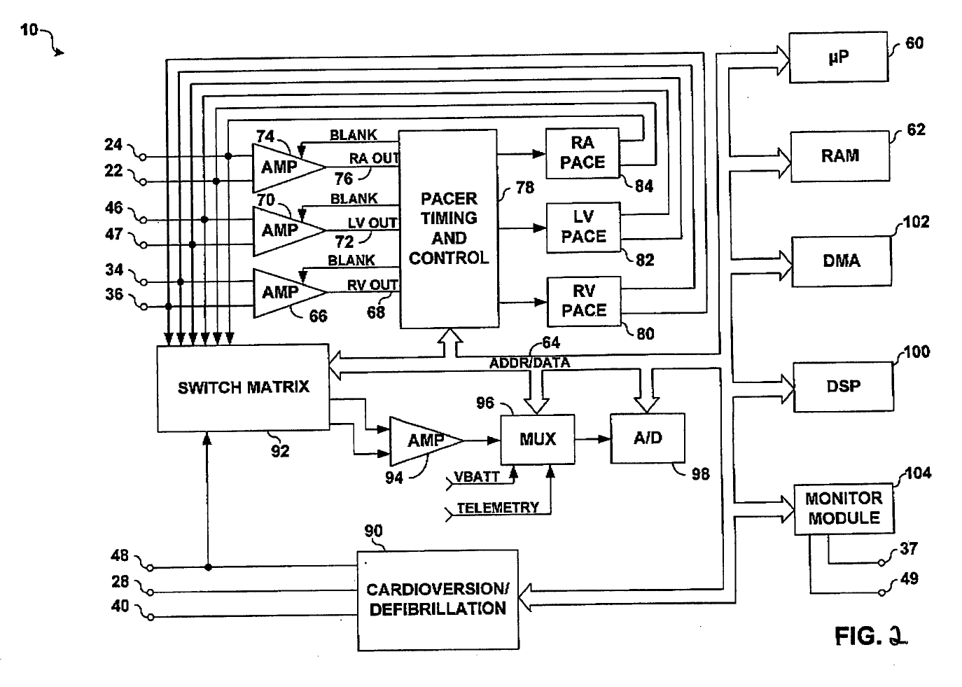

FIG. 2 is a functional block diagram of IMD 10. As shown in FIG. 2, IMD 10 may take the form of a multi-chamber ICD having a microprocessor-based architecture. However, this diagram should be taken as exemplary of the type of device in which various embodiments of the present invention may be embodied, and not as limiting, as it is believed that the invention may be practiced in a wide variety of device implementations, including devices that provide cardiac resynchronization pacing therapies but do not provide cardioverter and/or defibrillator functionality.

IMD 10 includes a microprocessor 60. Microprocessor 60 executes program instructions stored in a memory, e.g., a computer-readable medium, such as a ROM (not shown), EEPROM (not shown), and/or RAM 62. Program instruction stored in a computer-readable medium and executed by microprocessor 60, control microprocessor 60 to perform the functions ascribed to microprocessor 60 herein. Microprocessor 60 may be coupled to, e.g., to communicate with and/or control, various other components of IMD 10 via an address/data bus 64. IMD 10 paces heart 12. Pacer timing/control circuitry 78 preferably includes programmable digital counters, which control the basic time intervals associated with modes of pacing. Circuitry 78 also preferably controls escape intervals associated with pacing. For example, in some embodiments, IMD 10 paces right atrium 14 via timing/control circuitry 78 triggering generation of pacing pulses by pacer output circuit 84, which is coupled to electrodes 22 and 24. Pacer timing/control circuitry 78 may trigger generation of pacing pulses for right atrium 14 upon expiration of an atrial escape interval.

As mentioned above, IMD 10 provides a biventricular mode of cardiac resynchronization therapy. In exemplary embodiments, IMD 10 delivers pacing pulses to right ventricle 16 and left ventricle 17, separated by a V-V interval, to synchronize contractions of right ventricle 16 with contractions of left ventricle 17. Pacer timing/control circuitry 78 triggers generation of pacing pulses for right ventricle 16 by pacer output circuit 80, which is coupled to electrodes 34 and 36. Further, pacer timing/control circuitry 78 triggers generation of pacing pulses for left ventricle 17 by pacer output circuit 82, which is coupled to electrodes 46 and 47. The intervals to control CRT pacing, e.g., atrial escape, V-V, A-RV, and/or A-LV intervals, are maintained by circuitry 78, and the values of these intervals are provided to circuitry 78 by microprocessor 60 via bus 64. As will be described in greater detail below, in exemplary embodiments microprocessor 60 adjusts the values of at least some of these intervals based on a non-zero phase angle that indicates ventricular dysynchrony.

Circuitry 78 resets these intervals based on detection of paced and/or sensed depolarizations. Electrodes 34 and 36 are coupled to amplifier 66, which takes the form of an automatic gain controlled amplifier providing an adjustable sensing threshold as a function of measured signal amplitude. A signal is generated on RV out line 68 whenever the signal sensed between electrodes 34 and 36 exceeds the present sensing threshold. Thus, electrodes 34 and 36 and amplifier 66 are used to detect intrinsic right ventricular depolarizations.

Electrodes 46 and 47 are coupled to amplifier 70, which also takes the form of an automatic gain controlled amplifier providing an adjustable sensing threshold as a function of measured R-wave amplitude. A signal is generated on LV out line 22 whenever the signal sensed between electrodes 46 and 47 exceeds the present sensing threshold. Thus, electrodes 46 and 47 and amplifier 70 are used to detect intrinsic left ventricular depolarizations.

Electrodes 22 and 24 are coupled to amplifier 74, which takes the form of an automatic gain controlled amplifier providing an adjustable sensing threshold as a function of the measured P-wave amplitude. A signal is generated on RA out line 76 whenever the signal between electrodes 22 and 24 exceeds the present sensing threshold. Thus, electrodes 24 and 26 and amplifier 74 are used to detect intrinsic atrial depolarizations.

Output circuits 80, 82 and 84 are pulse generation circuits known in the art, which include capacitors and switches for the storage and delivery of energy as a pulse. In some embodiments, output circuits 80, 82 and 84 include circuitry for the delivery of current across combinations of electrodes 22, 24, 34, 36, 46, 47 and 52. Under the control of microprocessor 60, circuitry 78 directs the output circuits to deliver current via the selected electrodes. In exemplary embodiments, delivery of the current is controlled to occur for a cardiac cycle, beginning on a paced or sensed atrial event.

Microprocessor 60 uses switch matrix 92 to select which of available electrodes 22, 24, 34, 36, 46, 46 and 48 are coupled to amplifier 94 for use in digital signal analysis. The selected electrodes provide an impedance signal, and, in some embodiments, two or more impedance signals are digitally analyzed to determine the phase angle therebetween. However, IMD 10 as shown does not require digital signal analysis to determine the phase angle. Selection of electrodes is controlled by microprocessor 60 via data/address bus 66, and the selections may be varied as desired.

The analog signals derived from the selected electrodes and amplified by amplifier 94 are provided to multiplexer 96, and thereafter converted to a multi-bit digital signal by A/D converter 98. In exemplary embodiments, amplifier 94 filters noise from the signals according to a fundamental frequency (20 Hz or greater). In some embodiments, a digital signal processor (DSP) 100 process the multi-bit digital signals to identify time to onset of contraction, time to peak of contraction, and time to relaxation of contraction as will be described in greater detail below. In exemplary embodiments, microprocessor 60 executes mathematical algorithms to determine the phase angle between signals by determining a fundamental frequency phase for each signal, as will be described below. In some embodiments, the digital signals are stored in RAM 62 under control of direct memory access circuit 102 for later analysis by DSP 100 and/or microprocessor 60.

In other embodiments DSP 100 processes signals generated by sensors 37 and 49 to determine the phase angle therebetween. Sensors 37 and 49 are electrically coupled to monitor module 104 of IMD 10. Monitor module 104 includes various amplifiers, buffers, switches and the like to provide a filtered digital signal that reflects contraction of ventricles 16 and 17 to DSP 100. Although IMD 10 is described herein as having separate processors, microprocessor 60 may perform both the functions ascribed to it herein and digital signal analysis functions ascribed to DSP 100 herein. Moreover, although described herein in the context of microprocessor based ICD embodiment IMD 10, the invention may be embodied in various implantable medical devices that include one or more processors, which may be microprocessors, DSPs, FPGAs, or other digital logic circuits.

In some embodiments, IMD 10 detects ventricular and/or atrial tachycardias or fibrillations of heart 16 using tachycardia and fibrillation detection techniques and algorithms known in the art. For example, the presence of a ventricular or atrial tachycardia or fibrillation may be confirmed by detecting a sustained series of short R-R or P-P intervals of an average rate indicative of tachycardia, or an unbroken series of short R-R or P-P intervals. IMD 10 is also capable of delivering one or more anti-tachycardia pacing (ATP) therapies to heart 16, and cardioversion and/or defibrillation pulses to heart 16 via one or more of electrodes 48, 40 and 28.

In some embodiments, as shown in FIG. 2, Electrodes 48, 40 and 28, are coupled to a cardioversion/defibrillation circuit 90, which delivers cardioversion and defibrillation pulses under the control of microprocessor 60. Circuit 90 may include energy storage circuits such as capacitors, switches for coupling the storage circuits to electrodes 48, 40 and 28, and logic for controlling the coupling of the storage circuits to the electrodes to create pulses with desired polarities and shapes. Microprocessor 60 may employ an escape interval counter to control timing of such cardioversion and defibrillation pulses, as well as associated refractory periods.

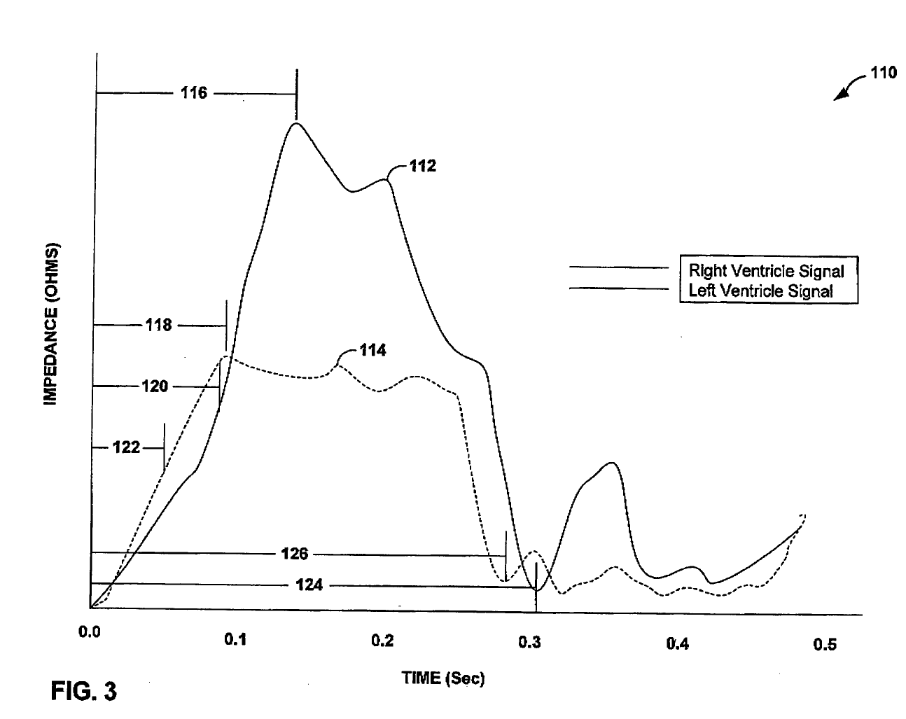

FIG. 3 is a timing diagram illustrating exemplary signals that reflect contractions of right atrium 16 and left atrium 17 of heart 12. Graph 110 contains a left ventricle signal 112 and a right ventricle signal 114. In particular, signals 112 and 114 are impedance signals that are measured, filtered, and A/D converted as described above with reference to FIGS. 1 and 2. However, as indicated above, the invention is not limited to digital processing of signals or the processing of impedance signals to evaluate ventricular synchronicity.

The large rise in impedance of both of signals 112 and 114 reflect a contraction of left ventricle 17 and right ventricle 16, respectively. According to graph 110 as shown in FIG. 3, the contraction of right ventricle 16 occurs prior to the contraction of left ventricle 17. In other words, the contractions of ventricles 16 and 17 illustrated by signals 112 and 114 are not synchronous.

IMD 10 determines a phase angle between signals 112 and 114 to evaluate ventricular dysynchrony. In some embodiments, microprocessor 60 utilizes a mathematical algorithm, described below, to determine the phase of each of signals 112, 114. In other embodiments, microprocessor 60 identifies fiducial points within each of signals 112 and 114, and compares the time of occurrence of the fiducial points within each of the signals to determine the phase angle. For example, DSP 100 can identify a left ventricular time to peak contraction 116 within left ventricular signal 112 and right ventricular time to peak contraction 118 within right ventricular signal 114. Microprocessor 60 compares right ventricle time to peak 118 and left ventricle time to peak 116 to determine the phase angle. Thus, as shown in FIG. 3, the phase angle when comparing time to peak 116 to time to peak 118 is a negative non-zero value.

In some embodiments, DSP 100 identifies other fiducial points within signals 112 and 114. These points are illustrated in graph 110 as times to onset 120 and 122 and times to relaxation 124 and 126. Time to onset 120 and 122 refer to a period of time until signals 112 and 114, respectively, reach a pre-specified threshold value indicative of an onset of left and right ventricular contractions, respectively. Times to relaxation 124 and 126 refer to a period of time until signal 124 and 126, respectively, reach a pre-specified value indicative of the relaxation of left and right ventricular contractions, respectively. In such embodiments, microprocessor 60 configures DSP 100 with the pre-specified threshold values used to identify these fiducial points.

In some embodiments, IMD 10 does not employ digital signal analysis to identify these fiducial points within signals 112 and 114. In such embodiments, IMD 10 includes analog peak, slope or threshold detecting amplifier circuits to identify the fiducial, as is known in the art. Further, in such embodiments, pacer timing/control circuit 78 receives the output of these amplifier circuits, and provides an indication of the occurrence of these events to microprocessor 60 so that microprocessor 60 is able to compare the times that these fiducial points occurred relative to the beginning of a cardiac cycle, e.g., an atrial depolarization, and determine the phase angle.

In exemplary embodiments, DSP 100 or microprocessor 60 mathematically processes signals 112 and 114 to determine their fundamental phase, and, by comparing the fundamental phases, to determine the phase angle between signals 112 and 114. The following exemplary mathematical computations (1)-(4) are used to determine the fundamental phase of a signal for an i^{th} cardiac cycle.
X=Σ_{k=1,N}ⁱcosⁱₖ(Zⁱₖ) (1)
Y=Σ_{k=1,N}ⁱsinⁱₖ(Zⁱₖ) (2)

Formulae (1) and (2) calculate X and Y which represent a sum of the cosine of all N impedance points (Zⁱₖ) and a sum of the sine of all N impedance points for the i^{th} cardiac cycle. Since both the first and second signals are sampled into N points the sum is necessary to compute the phase angle. Mathematically, equations (1) and (2) determine the inner product or projection.

Using results from equations (1) and (2) the phase of a signal is calculated as follows:
Pⁱ=atan2(Yⁱ/Xⁱ) (3)

Mathematically, equation (3) recovers the phase of the signal using the 4-quadrant arctangent function (atan2( )) to process the results of equations (1) and (2). Thus, the phase of the first signal (left ventricular phase, LVPⁱ) and the second signal (right ventricle phase, RVPⁱ) are calculated using equations (1)-(3).

The phase angle (φⁱ) for the i^{th} cardiac cycle between these two phases, also of the ith cardiac cycle, is calculated using equation (4) as follows.
φⁱ=RVPⁱ−LVPⁱ (4)

Thus, the phase angle is the difference between the phase of the second signal and the phase of the first signal. Further description of techniques for determining the fundamental phase of impedance signals can be found in U.S. Pat. No. 5,999,854, to Deno et al., which is incorporated herein by reference in its entirety. The phase angle between sensor signals may also be calculated in the this manner, or using fiducial points, for any of the types of sensor signals described above that can be received from sensors 37 and 49.

Based on the phase angle calculated by microprocessor 60 or received by microprocessor 60 from DSP 100, microprocessor 60 adjusts one or more timing intervals for ventricular pacing, such as the V-V timing interval, to reduce or eliminate this ventricular dysynchrony. Further, microprocessor 60 determines how to adjust one or more of the timing intervals based on whether the phase angle indicates that contraction of the right or left ventricle is occurring first, e.g., “leading.” For example, if the phase angle indicates that contraction of right ventricle 16 is leading contraction of left ventricle 17, microprocessor 60 adjusts the V-V interval, for example, to provide for earlier delivery of pacing pulses to left ventricle 17 relative to delivery of pacing pulses to right ventricle 16.

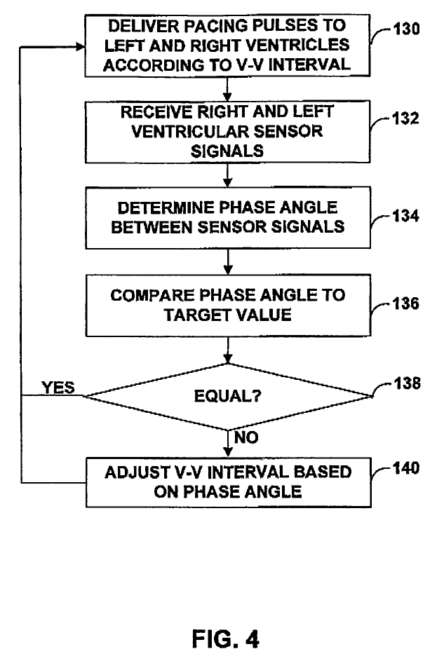

FIG. 4 is a flow chart illustrating an example method that IMD 10 may employ to deliver cardiac resynchronization therapy according to the invention. Pacer timing/control circuitry 78 controls delivery of pacing pulses to right and left ventricles 16 and 17 according to a first value of the V-V timing interval (130). Circuitry 78 can control delivery of the pacing pulses to the each of the ventricles 16 and 17 during the same or different cardiac cycles. Where the pulses are delivered during different cardiac cycles, circuitry 78 determines the timing of delivery of each pulse from an atrial depolarization, which is also used as a common reference point for comparison of sensor signals to determine the phase angle, as discussed above.

The pacing pulses result in contraction of ventricles 16 and 17. DSP 100 receives and processes sensor signals that reflect the contractions of ventricles 16 and 17, as described above (132). In some embodiments, the signals are received from sensors 37 and 49, which take the form of, for example, accelerometers, intracardiac pressure sensors, or oximeters. In exemplary embodiments, the signals are right and left ventricular impedance signals 112 and 114, generated as described above. In some embodiments where the signals are right and left ventricular impedance signals, IMD 10 delivers current between selected electrodes, described above with reference to FIG. 1, during the duration of the cardiac cycles in which the respective pacing pulses are delivered. The cardiac cycle can be defined as beginning on a sensed or paced atrial depolarization.

DSP 100 determines the phase angle between the signals using any of the techniques described above (134). For example, DSP 100 can determine the phase angle by comparing fiducial points of the signals or the fundamental frequency phases of the signals, as described above. DSP 100 provides the phase angle to microprocessor 60, which compares the phase angle to a predetermined target value, e.g., zero (136). If the phase angle is not equal to the target value (138), e.g., non-zero, microprocessor 60 determines an adjustment to one or more timing intervals for ventricular pacing, such as the V-V timing interval, and provides pacer timing/control circuitry 78 with an adjusted V-V interval (140). Microprocessor 60 determines the adjustment to the interval based on which of ventricles 16 and 17 the phase angle indicates is “leading,” The phase angle can be evaluated and in exemplary embodiments, the V-V timing intervals can be adjusted dynamically as described herein beat-to-beat, or periodically.

Various embodiments of the invention have been described. However, one skilled in the art will appreciate that various modifications can be made to these described embodiments without departing from the scope of the invention. For example, although described herein primarily in the context of determination of a signal phase angle between two signals, each of the signals associated with one of the left and right ventricles, the invention is not so limited.

In some embodiments, IMD 10 determines two or more phase angles between three or more signals. Further, in some embodiments, IMD 10 compares two or more signals associated with, e.g., measured from, a signal one of ventricles 16 and 17. In some embodiments, a signal used for determination of phase angles is a reference signal that is not associated with either of ventricles 16 and 17.

Moreover, the invention is not limited to adjust of a V-V interval. Various intervals associated with ventricular pacing, such as intervals between atrial events and delivery of pacing pulses to either of ventricles 16 and 17, an interval between an intrinsic depolarization of one of the ventricles and delivery of a pacing pulse to the other of the ventricles, or intervals between deliveries of two or more pacing pulses to the same one of ventricles 16 and 17 during the same cardiac cycle, can be adjusted based on one or more measured phase angles. These and other embodiments are within the scope of the following claims.

1. A method comprising:
receiving signals that reflect contractions of at least one ventricle of a heart, each of the signals received from a respective sensor implanted within a patient;
processing the signals to determine a phase angle between the signals; and
adjusting at least one interval for ventricular pacing based on the phase angle.

2. The method of claim 1, wherein receiving signals comprises:
receiving a first signal that reflects a contraction of a right ventricle of the heart; and
receiving a second signal that reflects a contraction of a left ventricle of the heart.

3. The method of claim 2, wherein receiving the first signal comprises:
delivering a first pacing pulse to the right ventricle; and
receiving the first signal in response to delivery of the first pacing pulse, and
wherein receiving the second signal comprises:
delivering a second pacing pulse to the left ventricle; and
receiving the second signal in response to delivery of the second pacing pulse.

4. The method of claim 3, wherein the first pacing pulse is delivered during a first cardiac cycle of the heart, and the second pacing pulse is delivered during a second cardiac cycle of the heart.

5. The method of claim 3, wherein the first and second pacing pulses are delivered during a single cardiac cycle.

6. The method of claim 3, wherein the first and second pacing pulses are delivered according to a value of the interval prior to adjustment.

7. The method of claim 1, wherein receiving signals comprises receiving impedance signals from electrodes implanted within the patient.

8. The method of claim 7, wherein the electrodes comprise a first and a second electrode, the first electrode is located approximately at the right ventricular apex of the heart, and the second electrode is located within a coronary sinus of the heart proximate to a free wall of a left ventricle of the heart.

9. The method of claim 1, wherein processing the signals to determine a phase angle between the signals comprises:
determining a fundamental frequency phase for each of the signals; and
comparing the fundamental frequency phases to determine the phase angle.

10. The method of claim 1, wherein determining the phase angle comprises:
identifying an occurrence of a feature within each of the signals;
identifying a time within a cardiac cycle for each of the signals based on the occurrence of the feature within that signal; and
comparing the times to determine the phase angle.

11. The method of claim 10, wherein the times comprise one of time to onset of contraction, time peak of contraction, and time to relaxation of contraction.

12. The method of claim 1, wherein the signals comprise one of accelerometer signals, intracardiac pressure signals, and intracardiac flow signals.

13. The method of claim 1, wherein adjusting at least one interval comprises adjusting a V-V interval between delivery of a first pacing pulse to a first ventricle of the heart and a second pacing pulse to a second ventricle of the heart.

14. The method of claim 1, wherein adjusting at least one interval comprises:
determining whether the adjustment to the interval exceeds a threshold value stored in a memory; and
adjusting the interval according to the determination.

15. A medical device comprising:
sensors implanted within a patient to generate respective signals that reflect contractions of at least one ventricle of a heart of the patient; and
a processor to determine a phase angle between the signals, and adjust at least one interval for ventricular pacing based on the phase angle.

16. The medical device of claim 15, wherein the sensors comprise a first sensor and a second sensor, the first sensor is located proximate to a right ventricle of the heart and generates a first signal the reflects a contraction of the right ventricle, and the second sensor is proximate to a left ventricle of the heart and generates a second signal that reflects a contraction of the left ventricle.

17. The medical device of claim 16, further comprising:
a first electrode to deliver a first pacing pulse to the right ventricle; and
a second electrode to deliver a second pacing pulse to the left ventricle, wherein the first sensor generates the first signal in response to delivery of the first pacing pulse, and the second sensor generates the second signal in response to delivery of the second pacing pulse.

18. The medical device of claim 17, wherein the processor controls delivery of the first pacing pulse during a first cardiac cycle, and controls delivery of the second pacing pulse during a second cardiac cycle.

19. The medical device of claim 17, wherein the processor controls delivery of the first and second pacing pulses during a single cardiac cycle.

20. The medical device of claim 17, wherein the processor controls delivery of the first and second pacing pulses according to a value of the interval prior to adjustment.

21. The medical device of claim 15, wherein the sensors comprise electrodes implanted within the patient to generate impedance signals that reflect contractions of the ventricles.

22. The medical device of claim 21, wherein the electrodes comprises a first and a second electrode, the first electrode is located approximately at the right ventricular apex of the heart, and the second electrode is located within a coronary sinus of the heart proximate to a free wall of a left ventricle of the heart.

23. The medical device of claim 15, wherein the processor determines a fundamental frequency phase for each of the signals, and compares the fundamental frequency phases to determine the phase angle.

24. The medical device of claim 15, wherein the processor identifies the occurrence of a feature within each of the signals, identifies a time within a cardiac cycle for each of the signals based on the occurrence of the feature within that signal, and compares the times to determine the phase angle.

25. The medical device of claim 24, wherein the times comprise one of time to onset of contraction, time peak of contraction, and time to relaxation of contraction.

26. The medical device of claim 15, wherein the sensors comprise one of flow sensors, accelerometers, pressure sensors, and oximeters.

27. The medical device of claim 15, wherein the processor adjusts a V-V interval between delivery of a first pacing pulse to a first ventricle of the heart and a second pacing pulse to a second ventricle of the heart based on the phase angle.

28. The medical device of claim 15, further comprising a memory to store a threshold value, wherein the processor determines whether the adjustment to the V-V interval exceeds the threshold value, and adjusts the V-V interval according to the determination.

29. The medical device of claim 15, wherein the medical device is implanted within the patient.

30. A computer-readable medium comprising instructions that cause a programmable processor to:
determine a phase angle between signals that reflect contractions of at least one ventricle of a heart of a patient, each of the signals generated by at least one sensor implanted within the patient; and
adjust at least one interval for ventricular pacing based on the phase angle.

31. The computer-readable medium of claim 30, wherein the instructions that cause a programmable processor to determine a phase angle comprise instructions that cause a programmable processor to:
process a first signal that reflects a contraction of a right ventricle of the heart; and
process a second signal that reflects a contraction of a left ventricle of the heart.

32. The computer-readable medium of claim 31, wherein the instructions that cause a programmable processor to process a first signal comprise instructions that cause a programmable processor to:
control delivery of a first pacing pulse to the right ventricle; and
process the first signal, the first signal generated in response to delivery of the first pacing pulse, and
wherein the instructions that cause a programmable processor to process a second signal comprise instructions that cause a programmable processor to:
control delivery of a second pacing pulse to the left ventricle; and
process the second signal generated in response to delivery of the second pacing pulse.

33. The computer-readable medium of claim 32, wherein the instructions that cause a programmable processor to control delivery of a first pacing pulse comprise instructions that cause a programmable processor to control delivery of the first pacing pulse during a first cardiac cycle of the heart, and the instructions that cause a programmable processor to control delivery of a second pacing pulse comprise instructions that cause a programmable processor to control delivery of the second pacing pulse during a second cardiac cycle of the heart.

34. The computer-readable medium of claim 32, wherein the instructions that cause a programmable processor to control delivery of first and second pacing pulses comprise instructions that cause a programmable processor to control delivery of the first and second pacing pulses during a single cardiac cycle of the heart.

35. The computer-readable medium of claim 32, wherein the instructions that cause a programmable processor to control delivery of first and second pacing pulses comprise instructions that cause a programmable processor to control delivery of the first and second pacing pulses according to a value of the interval prior to adjustment.

36. The computer-readable medium of claim 30, wherein the signals comprise impedance signals generated by electrodes implanted within the patient.

37. The computer-readable medium of claim 30, wherein the instructions that cause a programmable processor to determine a phase angle between the signals comprise instructions that cause a programmable processor to:
determine a fundamental frequency phase for each of the signals; and
compare the fundamental frequency phases to determine the phase angle.

38. The computer-readable medium of claim 30, wherein the instructions that cause a programmable processor to determine a phase angle comprise instructions that cause a programmable processor to:
identify an occurrence of a feature within each of the signals;
identify a time within a cardiac cycle for each of the signals based on the occurrence of the feature within that signal; and
compare the times to determine the phase angle.

39. The computer-readable medium of claim 38, wherein the times comprise one of time to onset of contraction, time peak of contraction, and time to relaxation of contraction.

40. The computer-readable medium of claim 30, wherein the signals comprise one of accelerometer signals, intracardiac pressure signals, and intracardiac flow signals.

41. The computer-readable medium of claim 30, wherein the instructions that cause a programmable processor to adjust an interval comprise instructions that cause a programmable processor to adjust a V-V interval between delivery of a first pacing pulse to a first ventricle of the heart and a second pacing pulse to a second ventricle of the heart based on the phase angle.

42. A medical device comprising:
means for generating signals that reflect contractions of at least one ventricle of a heart; and
means for determining a phase angle between the signals, and adjusting at least one interval for ventricular pacing based on the phase angle.

43. The medical device of claim 42, wherein the means for generating signals comprises:
means for generating a first signal that reflects a contraction of a right ventricle of the heart; and
means for generating a second signal that reflects a contraction of a left ventricle of the heart.

44. The medical device of claim 43, further comprising:
means for delivering a first pacing pulse to the right ventricle; and
means for delivering a second pacing pulse to the left ventricle, wherein the means for generating the first signal generates the first signal in response to delivery of the first pacing pulse, and the means for generating a second signal generates the second signal in response to delivery of the second pacing pulse.

45. The medical device of claim 44, further comprising means for controlling the first and second delivery means to deliver the first and second pacing pulses according to a value of the interval prior to adjustment.

46. The medical device of claim 42, wherein means for generating signals comprises means for generating intracardiac impedance signals.

47. The medical device of claim 42, wherein the means for determining a phase angle between the signals comprises:
means for determining a fundamental frequency phase for each of the signals; and
means for comparing the fundamental frequency phases to determine the phase angle.

48. The medical device of claim 42, wherein the means for adjusting at least one interval comprises means for adjusting a V-V interval between delivery of a first pacing pulse to a first ventricle of the heart and a second pacing pulse to a second ventricle of the heart based on the phase angle.
